SEQUENCE LISTING (2 sequences)

SEQ ID NO: 1 - protein, 491 aa, Homo sapiens [sequence omitted]

SEQ ID NO: 2 - protein, 12 aa, synthetic construct
  REGION 1..12: note = colonic-targeting peptide which is generated from the COOH-terminal globular domain belonging to the laminin-1 alpha1 chain
TWYKIAFQRNRK

(19) United States
(12) Patent Application Publication
(10) Pub. No.: US 2024/0299505 A1
(43) Pub. Date: Sep. 12, 2024

(54) COMPOSITION FOR IMPROVING, PREVENTING OR TREATING INFLAMMATORY BOWEL DISEASE COMPRISING NAMPT-DERIVED PEPTIDE AS ACTIVE INGREDIENT

(71) Applicant: Industry-University Cooperation Foundation Hanyang University ERICA Campus, Gyeonggi-do (KR)

(72) Inventors: Chul Su YANG, Gyeonggi-do (KR); Hyo Keun KIM, Gyeonggi-do (KR)

(21) Appl. No.: 18/523,363

(22) Filed: Nov. 29, 2023

(30) Foreign Application Priority Data
Mar. 8, 2023 (KR) 10-2023-0030495

(51) Int. Cl. A61K 38/45; A61P 1/00; C12N 9/10

(52) U.S. Cl. CPC A61K 38/45; A61P 1/00; C12N 9/1077; C12Y 204/02012

(57) ABSTRACT

Provided is a composition for improving, preventing, or treating inflammatory bowel disease including a NAMPT-derived peptide as an active ingredient. The NAMPT-derived peptide of the present disclosure may bind to TRL4 and/or CYBB competitively with extracellular NAMPT (eNAMPT) to inhibit NLRP3 inflammasome activation by the interaction of eNAMPT with TRL4 and/or CYBB, and the NAMPT-derived peptide of the present disclosure further includes a peptide targeting colon tissue to act directly on the colon, so that it is expected to be useful for the prevention or treatment of inflammatory bowel disease.

### CROSS-REFERENCE TO RELATED APPLICATION

This application claims the benefit of Korean Patent Application No. 10-2023-0030495 filed on Mar. 8, 2023, in the Korean Intellectual Property Office, the entire disclosure of which is incorporated herein by reference for all purposes.

### SEQUENCE LISTING

The instant application contains a Sequence Listing which has been submitted in .xml format and is hereby incorporated by reference in its entirety. Said .xml file is named “083972_00030_ST26.xml”, was created on Nov. 27, 2023, and is 3,915 bytes in size.

### BACKGROUND

### 1. Field of the Invention

The present disclosure relates to a composition for improving, preventing, or treating inflammatory bowel disease including a NAMPT-derived peptide as an active ingredient.

### 2. Description of the Related Art

Inflammatory bowel diseases (IBDs), including Crohn's disease as well as ulcerative colitis (UC), are characterized by abnormalities in intestinal homeostasis, which result in chronic inflammation. Biological drugs (anti-TNFα therapy and/or immune modulators) are effective for treating many people with IBD. However, many patients with severe disease fail to achieve effective treatment with existing drugs because of a poor responsiveness to drugs, unavailability of mechanism-specific targeted therapy, or serious adverse effects. Accordingly, highly sensitive and specific predictive biomarkers along with novel therapeutic strategies are required to implement effective treatment strategies.

Nicotinamide phosphoribosyl transferase (NAMPT) acts as a catalyst of the rate-determining step of a nicotinamide adenine dinucleotide (NAD) recovery pathway to regulate intracellular NAD concentrations. There are many evidences indicating that many types of cells, including monocytes and macrophages, secrete NAMPT, and extracellular (e)-NAMPT (visfatin) acts as a novel soluble factor with similar activity to cytokines, adipokines, and DAMPs. However, the role of (e)NAMPT in inflammatory macrophages in ulcerative colitis is not fully known.

The present inventors confirmed that overexpression of NAMPT and CYBB in patients with ulcerative colitis, and interaction of eNAMPT with TLR4 and/or CYBB induced chronic activation of NLRP3 inflammasome, developed a NAMPT-derived peptide capable of inhibiting the interaction between TLR4 and/or CYBB and eNAMPT, confirmed the NLRP3 inflammasome-mediated inflammatory response inhibitory activity and a therapeutic effect in a DSS-induced colitis animal model of the NAMPT-derived peptide, and then completed the present disclosure.

### SUMMARY

An aspect of the present disclosure is to provide a pharmaceutical composition for preventing or treating inflammatory bowel disease including a NAMPT-derived peptide capable of interacting with TLR4 and/or CYBB as an active ingredient.

In addition, another aspect of the present disclosure is to provide a health functional food composition for preventing or improving inflammatory bowel disease including the NAMPT-derived peptide as an active ingredient.

However, technical aspects of the present disclosure are not limited to the aforementioned purpose and other aspects which are not mentioned may be clearly understood to those skilled in the art from the following description.

According to an aspect, there is provided a pharmaceutical composition for preventing or treating inflammatory bowel disease including a nicotinamide phosphoribosyl transferase (NAMPT)-derived peptide as an active ingredient.

In addition, according to another aspect, there is provided a health functional food composition for preventing or improving inflammatory bowel disease including the NAMPT-derived peptide as an active ingredient.

In an embodiment of the present disclosure, the NAMPT is an enzyme consisting of SEQ ID NO: 1, and the NAMPT-derived peptide may include or consist of one or more amino acid sequences selected from the group consisting of amino acids 57 to 65, amino acids 52 to 56, and amino acids 52 to 65 in the amino acid sequence of NAMPT.

In another embodiment of the present disclosure, the N-terminus or C-terminus of the NAMPT-derived peptide may bind to a laminin α-1-derived peptide, and the binding means including the NAMPT-derived peptide in which the laminin α-1-derived peptide directly binds to the N-terminus or C-terminus or indirectly to the N-terminus or C-terminus through a linker.

In another embodiment of the present disclosure, the laminin α-1-derived peptide may have affinity for integrin α6β1 and may include or consist of an amino acid sequence represented by SEQ ID NO: 2.

In another embodiment of the present disclosure, when the NAMPT-derived peptide includes amino acids 57 to 65 in the amino acid sequence of NAMPT, the NAMPT-derived peptide may bind to TLR4, and when the NAMPT-derived peptide includes amino acids 52 to 56 in the amino acid sequence of NAMPT, the NAMPT-derived peptide may bind to CYBB. Accordingly, when the NAMPT-derived peptide includes amino acids 52 to 65 in the NAMPT amino acid sequence, the NAMPT-derived peptide may bind to TLR4 and CYBB.

In another embodiment of the present disclosure, the NAMPT-derived peptide may inhibit activation of NLRP3 inflammasome, and this is because the interaction of eNAMPT with TLR4 and/or CYBB, which induces the activation of the NLRP3 inflammasome, is inhibited by the NAMPT-derived peptide. That is, the NAMPT-derived peptide competitively interacts with eNAMPT and TLR4 and/or CYBB to inhibit the activation of the NLRP3 inflammasome.

In another embodiment of the present disclosure, the NAMPT-derived peptide may reduce NLRP3 inflammasome-mediated inflammation.

In addition, according to yet another aspect, there is provided a method for preventing or treating inflammatory bowel disease including administering the NAMPT-derived peptide to a subject.

In addition, according to yet another aspect, there is provided a use of the NAMPT-derived peptide for preparing a drug for preventing or treating inflammatory bowel disease.

According to the present disclosure, the NAMPT-derived peptide may bind to TRL4 and/or CYBB competitively with extracellular NAMPT (eNAMPT) to inhibit NLRP3 inflammasome activation by the interaction of eNAMPT with TRL4 and/or CYBB, and the NAMPT-derived peptide of the present disclosure further includes a peptide targeting colon tissue to act directly on the colon, and thus may be useful for the prevention or treatment of inflammatory bowel disease.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1A illustrates association of NAMPT with NLRP3 inflammasome activation driven by TLR4 or NADPH oxidase;

FIG. 1B illustrates violin plots for the expression of key genes in macrophages associated with NLRP3 inflammasome activation in SCP259;

FIG. 1C illustrates violin plots of the expression of key genes associated with NLRP3 inflammasome activation in GSE182270;

FIG. 2 illustrates results of an experiment of confirming direct binding of eNAMPT to CYBB or TLR4.

Specifically, (A) normal human cases along with UC patients were used for IB with αNAMPT and αCYBB. For IB with αActin (upper), whole cell lysates (WCLs) were used. Representative gel images derived from five independent healthy controls and patients were shown. The expression of NAMPT mRNA and CYBB mRNA was measured by quantitative real-time PCR (lower). Five of ten data from normal human and UC patients were shown. Statistical significance was evaluated by the Student's t-test coupled with the Bonferroni adjustment (***p<0.001) compared to human normal. (B) The colons of normal mice and mice with acute and chronic colitis were analyzed for IB with αNAMPT and αCYBB. WCLs were used for IB together with αActin. Biological replicates (n=10) for each condition were performed. (C) BMDMs were activated with LPS (100 ng/mL) for the indicated times (left), primed with LPS (100 ng/ml) for 4 hours, and then stimulated with ATP (1 mM) for the indicated times (right). IB in the supernatant (SN) with αNAMPT and WCL with αNAMPT or αActin. (D, E) BMDMs were primed with LPS and stimulated with ATP (left) or incubated with rNAMPT (1 μg/mL) for 2 hours. BMDMs were treated for IP with αNAMPT or αHis, and then treated sequentially IB with αTLR4, αCYBB, or αCYBA (D), and IB with αASC or αNLRP3(E). WCLs were used for IB with αTLR4, αCYBB, αCYBA, αASC, αNLRP3 or αActin. (F) Titration of fluorescently labeled TLR4 or CYBB with unlabeled NAMPT, using curve fit analysis to determine Kd (219 and 896 nM). The data come from five independent experiments that yielded comparable results;

FIG. 3 illustrates experimental design and results for identifying an essential region of NAMPT for binding to TLR4 and CYBB. Specifically, (A) Schematic diagram (upper) of the structures of NAMPT and TLR4. 293T cells were transfected with GST-NAMPT or its mutants and Flag-TLR4. GST pulldown was performed with 293T cells and then IB was performed using αFlag. WCLs were used for IB using αGST, αFlag, or αActin (lower, left in A). 293T cells were transfected with Flag-TLR4 or its mutants and Myc-NAMPT. The 293T cells were used for IP with αMyc, followed by IB with αFlag. WCLs were used for IB using αMyc, αFlag or αActin (lower, right in A). (B) 293T cells were transfected with Myc-NAMPT and Flag-TLR4 and treated with NAMPT peptide or its mutants for 6 hours (5 μM, left; 1, 5, 10 μM, right). The 293T cells were used for IP with αFlag, followed by IB with αMyc. WCLs were used for IB using αMyc, αFlag, or αActin. (C) Schematic diagram (upper) of the structures of CYBB. 293T cells were transfected with GST-NAMPT or its mutants and V5-CYBB (lower, left in C). The 293T cells were transfected with GST-CYBB or its mutants and Myc-NAMPT (lower, right in C). The 293T cells were subjected to GST pull-down, followed by IB with αV5 or αMyc. WCLs were used for IB using αGST, αMyc, a V5 or αActin. (D) 293T cells were transfected with Myc-NAMPT and V5-CYBB and treated with NAMPT peptide or its mutants for 6 hours (5 μM, left; 1, 5, 10 μM, right). The 293T cells were used for IP with a V5, followed by IB with αMyc. WCLs were used for IB using αMyc, αFlag, or αActin. (E) BMDMs were incubated with rNAMPT (1, 5, and 10 μg/mL) for 2 hours. BMDMs were subjected to IP with αHis, followed by IB with αTLR4, αCYBB, or αCYBA. WCLs were used for IB together with αTLR4, αCYBB or αCYBA, and αHis or a Actin. Data come from seven independent experiments that yielded comparable results (A-E). Ref star: Binding domain with binding partners;

FIG. 4 confirms that NAMPT-derived peptides inhibit activation of signal 1 and 2 of NLRP3. Specifically, (A-C) BMDMs were stimulated with LPS for 4 hours and pretreated with rNAMPT for 1 hour (A, B) or 18 hours (C). (A) WCLs were used for IB with αIkB-α, αP-IkB-α, or αActin. (B) FACS analysis for H₂O₂ (probe for 2′,7′-dichlorofluorescin diacetate) or O²⁻ (probe for dihydroethidium). Quantitative analysis (upper) of mean fluorescence intensities of H₂O₂ and O²⁻ and NADPH oxidase activity (lower), (C) Culture supernatants (SN) were harvested and analyzed for cytokine ELISA. (D, E) LPS-primed BMDMs were treated with rNAMPT for 1 hour and then activated with ATP for 30 minutes. (D) ELISA for IL-1β and IL-18. (E) IB in SN with αIL-1β p17, αIL-18 p18, or αCasp1 p10 and IB of WCL with αPro-IL-1β, αPro-IL-18, αPro-Casp1, or αActin. The data come from seven independent experiments that yielded comparable results (A, E). Data come from three independent experiments that yielded similar results. (B-D) Data are provided as mean±SD of three experiments. Significance difference compared to LPS+PBS (*p<0.05; **p<0.01; ***p<0.001);

FIG. 5 illustrates results confirming an effect of TAT-NAMPT or NAMPT peptide on inflammation. Specifically, (A) BMDMs were stimulated with LPS (100 ng/ml) for 18 hours and pretreated with rNAMPT for 1 hour. Culture supernatants were collected and analyzed for cytokine ELISA. (B) LPS-primed BMDMs were treated with rNAMPT for 1 hour and then activated with nigericin for 30 minutes and DSS for 24 hours. ELISA for IL-1B and IL-18. Data showed three independent experiments having similar results. The shown data are the mean±SD of three experiments. Significant difference comparing LPS+PBS (*p<0.05; **p<0.01; ***p<0.001);

FIG. 6A illustrates a schematic diagram (upper) of rCT-NAMPT, results of analyzing 6×His-rCT-NAMPT, rVehicle or rCT purified by bacteria using IB with Coomassie blue staining or αHis (lower left) and cell viability of BMDMs treated with rVehicle, rCT or rCT-NAMPT for the indicated period and concentration determined using an MTT test (lower right);

FIG. 6B illustrates results of constructing a DSS-induced model for colitis, in which mice were fed with 3% DSS for 6 days and evaluated on Day 6 (left). The collected colon was used for analysis of the number of ITGA6+ or ITGB1+ cells by IB and FACS using αITGA6, αITGB1, or αActin;

FIG. 6C illustrates a scheme of an acute colitis model introduced with Lenti-shNS or Lenti-shITG virus and treated with 3% DSS, and a result of performing IB using intraperitoneal administration of rCT-NAMPT conjugated with Cy5.5 for 1 hour and αITGA6, αITGB1, or αActin in the collected colon. The number of ITGA6+ or ITGB1+ cells was analyzed by FACS.

FIG. 7 illustrates results of analyzing the number of ITGA6+ or ITGB1+ cells in the colon collected as an extension of the experiment on the effect of r Vehicle peptide on colon targeting by IB and FACS using αITGA6, αITGB1, or αActin;

FIG. 8 confirms an effect of rCT-NAMPT in mice with DSS-induced acute colitis. Specifically, (A) an experimental plan for an acute colitis model treated with rCT-NAMPT (50 μg/kg) and 3% DSS after introduction of Lenti-shNS or Lenti-shITG virus (left) and the survival rates of mice monitored for 12 days (right). Mortality was measured with n=15 mice per group. Statistical differences between rVehicle treated animals were recorded (log-rank test). These data come from two different experiments that yielded comparable results. (B) confirms a change in body weight of the experimental animals during an experiment period in (A) above (n=8). (C) is a colitis score result of the experimental animals in (A) above. Colitis scores were obtained from clinical parameters (weight loss, stool concentration, bleeding) (n=8). (D) Photograph (upper) and length (lower) of the colon in mice treated with rVehicle, rCT, or rCT-NAMPT (n=8) and 3% DSS. (E) Results of performing IP and IB using the colon tissue. Colon tissue was used for IP with αHis or αNAMPT and then for IB with αTLR4, αCYBB, or αCYBA. WCLs were used for IB together with αTLR4, αCYBB or αCYBA, and αHis or αActin. (F) Results of FACS analysis (n=8) of ROS in cells collected from the colon. (G) Cytokine and MPO activity levels in colon homogenates (n=10). (H) Results of hematoxylin and eosin (H&E) staining of the colon (left) (n=10): Representative radiohistopathology scores were evaluated using rVehicle, rCT, or rCT-NAMPT in 3% DSS-treated mice as illustrated in techniques (Materials and Methods). Statistical significance was evaluated using a Student's t-test using Bonferroni correction (***p<0.001) in comparison to rVehicle;

FIG. 9 confirms an effect of rCT-NAMPT on acute DSS-induced colitis in TLR4−/− and CYBB−/− mice. Specifically, a scheme of the acute colitis model was treated to rCT-NAMPT (50 μg/kg) and 3% DSS (upper). The survival of mice was tracked for 12 days, and mortality was calculated for n=15 mice per group. Statistical differences between r Vehicle treated mice were shown (log-rank test). Data showed two independent experiments having similar results. Colitis scores were obtained from clinical parameters (weight loss, stool consistency, bleeding) (n=8, or less); and

FIG. 10 confirms an effect of rCT-NAMPT in mice with DSS-induced chronic colitis. Specifically, (A) Scheme of the chronic colitis model treated with rCT-NAMPT (50 μg/kg) and 3% DSS. (B) The survival of mice was tracked for 9 weeks, and mortality was calculated for n=15 mice per group. Statistical differences between rVehicle treated animals were recorded (log-rank test). These data come from two different experiments that yielded comparable results. (C) Weight loss of rVehicle, rCT or rCT-NAMPT treatment in mice was confirmed (n=15). (D) Image (upper) and length (lower) of colon in 3% DSS-induced chronic colitis mice with rVehicle, rCT or rCT-NAMPT. (E) H&E staining of the colon (left) (n=8): a representative image. H&E staining was used to evaluate histopathology scores in 3% DSS-induced chronic colitis mice treated with rVehicle, rCT, or rCT-NAMPT. The Student's t-test with the Bonferroni adjustment was used to establish statistical significance when compared to r Vehicle (***p<0.001).

### DETAILED DESCRIPTION

Nicotinamide phosphoribosyl transferase (NAMPT) regulates intracellular NAD concentration, but extracellular NAMPT is known to be mainly involved in inflammation mediated by macrophages.

Hereinafter, in the present disclosure, extracellular NAMPT is referred to as eNAMPT due to a difference in the action of NAMPT inside and outside a cell.

Through analysis of single-cell RNA-seq data, the present inventors confirmed that the levels of NAMPT and CYBB/NOX2 in macrophages were increased in colitis patients and mouse models of acute and chronic colitis. From the above, it was expected that NAMPT and CYBB would be clinically important in colitis. In particular, cellular and serum levels of eNAMPT were increased in patients with IBD who are unresponsive to anti-TNFα treatment agent (adalimumab or infliximab), and eNAMPT levels were decreased in responsive patients to values comparable with those of healthy controls. The findings suggest that this enzyme may serve as a potential target for drugs to effectively and safely treat IBD because eNAMPT levels are correlated with worse prognosis.

From the results, the present inventors confirmed the mechanisms of intracellular and extracellular NAMPT in inflammatory responses. In macrophages, eNAMPT directly interacted with CYBB to increase production of ROS and activation of the NLRP3 inflammasome along with activation of the TNF-α and NF-κB signaling pathways through direct stimulation by eNAMPT-TLR4 interaction.

From the above, eNAMPT interacts with CYBB and TLR4 to induce ROS production and activation of the NLRP3 inflammasome. Therefore, the strategy was to inhibit the interaction of eNAMPT with CYBB or TLR4, and a region of eNAMPT that interacts with CYBB and TLR4 was identified. It was confirmed that a 57-65 a.a. region of NAMPT was the minimum region that binds to TLR4, and a 52-56 a.a region of NAMPT was the minimum region that binds to CYBB. Peptides consisting of the regions that bind to the identified NAMPT and TLR4 or CYBB were prepared, and it was confirmed that each peptide inhibited an NLRP3 inflammasome-mediated inflammatory response.

Accordingly, the present inventors provide a NAMPT-derived peptide capable of interacting with CYBB and/or TLR4 for the prevention or treatment of inflammatory bowel disease.

Meanwhile, intensive research focusing on colonic-targeted drug delivery systems for topical treatment of colonic diseases shows that colonic-targeted drug delivery systems cause fewer systemic side effects and improve the effectiveness of oral delivery of therapeutic agents susceptible to acidic and enzymatic breakdown in the upper gastrointestinal tract.

Thus, the present inventors targeted inflamed colon by linking a 12-residue peptide (TWYKIAFQRNRK; designated “CT” for colonic-targeting) to the NAMPT-derived peptide in a COOH-terminal globular domain belonging to a laminin-1 al chain with high affinity with the integrin α6β1.

Therefore, the NAMPT-derived peptide of the present disclosure may be provided in a form bound to the CT peptide, the binding of the NAMPT-derived peptide and the CT peptide may also mean that the CT peptide may be directly linked to the N-terminus or C-terminus of the NAMPT-derived peptide, and indirectly linked through a linker without affecting the structures and functions of the NAMPT-derived peptide and the CT peptide.

In the present disclosure, the NAMPT-derived peptide bound to the CT peptide is indicated as “CT-NAMPT”.

CT-NAMPT targeted the inflamed colon in vitro and in vivo and inhibited the activation of the NLRP3 inflammasome. Furthermore, it was confirmed that CT-NAMPT increased the survival rate of mice and reduced the levels of ROS and cytokines in the colon in animal models with DSS-induced acute and chronic colitis to verify the possibility as a therapeutic agent of the CT-NAMPT for inflammatory bowel disease.

As used in the present disclosure, the “NAMPT-derived peptide” may include or consist of a 57-65 amino acid region of a NAMPT protein consisting of an amino acid sequence represented by SEQ ID NO: 1, may include or consist of a 52-56 amino acid region of the NAMPT protein, and may include or consist of a 52-65 amino acid region of the NAMPT protein.

As used herein, the NAMPT-derived peptide may be used interchangeably with the term “NAMPT peptide”.

As used herein, the term “peptide” refers to a linear molecule formed by binding amino acid residues with each other by peptide bonds. The NAMPT-derived peptide of the present disclosure may be obtained by fragmenting the NAMPT protein, and may be prepared according to chemical synthesis methods known in the art, especially solid-phase synthesis technology or liquid-phase synthesis technology.

The NAMPT-derived peptide of the present disclosure may induce modifications of an amino (N—) terminus or a carboxy (C—) terminus in order to increase its activity. Through these modifications, the peptide of the present disclosure may have an increased half-life upon in vivo administration. For example, the amino terminus of the NAMPT-derived peptide may be bound with a protecting group, such as an acetyl group, a fluorenyl methoxycarbonyl group, a formyl group, a palmitoyl group, a myristyl group, a stearyl group, and polyethylene glycol (PEG), and the carboxy terminus of the peptide may be modified with a hydroxyl group (—OH), an amino group (—NH₂), an azide (—NHNH₂), or the like. In addition, the terminus of the peptide of the present disclosure or an R-residue (R-group) of the amino acid may be bound with fatty acids, oligosaccharides chains, all nanoparticles (gold particles, liposomes, heparin, hydrogel, etc.), amino acids, carrier proteins, and the like. The modification of the amino acids described above serve to improve the potency and stability of the peptide of the present disclosure. As used herein, the term “stability” refers not only to in vivo stability, but also storage stability (including storage stability at room temperature, refrigeration, and frozen storage).

In the present disclosure, the “inflammatory bowel disease” refers to a disease in which abnormal chronic inflammation in the intestinal tract repeats improvement and recurrence, and includes ulcerative colitis and Crohn's disease.

In the present disclosure, the “treatment” means all actions that improve or advantageously change symptoms of the inflammatory bowel disease by the administration of the pharmaceutical composition according to the present disclosure.

In the present disclosure, the “prevention” means all actions that inhibit inflammatory bowel disease or delay the onset of the inflammatory bowel disease by administration of the composition according to the present disclosure.

In the present disclosure, the “improvement” means all actions that at least reduce parameters associated with conditions to be treated, e.g., the degree of symptoms. At this time, the health functional food composition may be used simultaneously or separately with a drug for treatment, before or after the onset of the corresponding disease in order to prevent or improve the inflammatory bowel disease.

In the present disclosure, the “subject” is not limited as long as it is a mammal, but may desirably be a human or livestock.

In the present disclosure, the pharmaceutical composition may further include one or more known treatments for inflammatory bowel disease in addition to the NAMPT-derived peptide, and may further include suitable carriers, excipients, and diluents commonly used in the preparation of the pharmaceutical composition.

In the present disclosure, the “carrier” is also called a vehicle, and refers to a compound that facilitates the addition of peptides into cells or tissues, and for example, dimethylsulfoxide (DMSO) is a commonly used carrier that facilitates the injection of many organic substances into the cells or tissues of living organisms.

In the present disclosure, the “diluent” is defined as a compound diluted in water that not only stabilizes a biologically active form of a target peptide, but also dissolves the protein or peptide. Salts dissolved in a buffer solution are used as diluents in the art. A commonly used buffer solution is phosphate buffered saline because the buffer solution imitates a salt state of a human solution. Since the buffer salt may control the pH of the solution at a low concentration, the buffer diluent rarely modifies the biological activity of the compound. Compounds containing azelaic acid used herein may be administered to human patients by themselves or as a pharmaceutical composition mixed with other ingredients or with suitable carriers or excipients, like combination therapy.

In addition, the pharmaceutical composition for preventing or treating inflammatory bowel disease including the NAMPT-derived peptide according to the present disclosure as an active ingredient may be formulated and used in the form of powders, granules, tablets, capsules, suspensions, emulsions, syrups and sterile injectable solutions according to conventional methods. The composition of the present disclosure may be administered orally or parenterally (e.g., applied intravenously, subcutaneously, intraperitoneally, or topically) depending on an intended method. The dose varies depending on the condition and weight of a patient, the degree of a disease, a drug form, and administration route and period, but may be appropriately selected by those skilled in the art. For example, the dose may be administered in 0.001 mg to 1000 mg in the form mixed with the pharmaceutically acceptable carrier. The composition of the present disclosure may be administered once or several times a day as needed, and may be used alone or in combination with surgery, hormone therapy, drug therapy, and methods using biological response regulators.

In addition, the present disclosure provides a food composition or feed composition including a NAMPT-derived peptide as an active ingredient. When using the NAMPT-derived peptide of the present disclosure as an additive to food or feed, the NAMPT-derived peptide may be added as it is or used together with other foods, feeds, or ingredients thereof, and may be used appropriately according to conventional methods. The mixing amount of the active ingredients may be appropriately determined depending on the purpose of use. In general, the NAMPT-derived peptide of the present disclosure is added in an amount of 15 wt % or less, desirably 10 wt % or less, based on the raw materials when preparing feeds, foods or beverages. However, in the case of long-term intake for the purpose of health and hygiene or for the purpose of health control, the amount may be equal to or less than the range, and there is no problem in terms of safety, so that the active ingredients may be used even in an amount above the range. The types of food and feed are not particularly limited.

In the present disclosure, an amino acid sequence is abbreviated as follows according to the IUPAC-IUB nomenclature.

Arginine (Arg, R), Lysine (Lys, K), histidine (His, H), serine (Ser, S), threonine (Thr, T), glutamine (Gln, Q), asparagine (Asp, N), methionine (Met, M), leucine (Leu, L), isoleucine (Ile, I), valine (Val, V), phenylalanine (Phe, F), tryptophan (Trp, W), tyrosine (Tyr, Y), alanine (Ala, A), glycine (Gly, G), proline (Pro, P), cysteine (Cys, C), aspartic acid (Asp, D), glutamic acid (Glu, E), norleucine (Nle)

The present disclosure may have various modifications and various embodiments and specific embodiments will be illustrated in the drawings and described in detail in the detailed description. However, the present disclosure is not limited to specific embodiments, and it should be understood that the present disclosure covers all the modifications, equivalents and replacements within the idea and technical scope of the present disclosure. In the interest of clarity, not all details of the relevant art are described in detail in the present specification in so much as such details are not necessary to obtain a complete understanding of the present disclosure.

### Methods and Materials

### 1. Analysis of Single-Cell RNA-Seq Data for Ulcerative Colitis

### 1-1. Dataset

In this study, in order to secure sufficient information on macrophages and T cells, in SCP259 (Smillie, C. S. et al., Cell 2019, 178, 714-730) and GSE182270 (Uzzan, M. et al., Nat. Med. 2022, 28, 766-779) datasets, data applicable to the human cell atlas was used (Table 1). Since the number of macrophages was limited in GSE182270, the data was used as supplementary data for DEG and GSE analysis to verify the main results of SCP259.

TABLE 1

| | | Condition and Number of Samples | | | Total Number | Cell Type |
|---|---|---|---|---|---|---|
| Dataset | Tissue | Healthy | Inflamed | Non-Inflamed | of Cells | Annotation |
| SCP259 | Colon | 12 | 18 | 18 | 365 K | Available |
| | (LP and Epi) | | | | | |
| GSE182270 | Colon (LP) | 4 | 5 | — | 32 K | Not available |

LP: lamina propria, Epi: Epithelium.

### 1-2. DEG and GSEA

Differentially expressed gene (DEG) analysis and gene set enrichment analysis (GSEA) were performed individually on the selected cells for the two datasets described above. Specifically, macrophages, regulatory T cells, CD4+ T cells and cytotoxic T cells were selected and analyzed. The latest version of Seurat package was used for the DEG analysis and FGSEA package was used for GSEA.

Since the number of cells in GSE182270 was much smaller than that in SCP259, the analysis was performed as follows (1) to (3).
(1) DEG: DEGs were identified separately for the two datasets with a p-value cutoff of 0.05. A non-adjusted p-value was used to obtain as many DEGs as possible.
(2) Selection of common DEGs: Thereafter, DEGs having the same sign of log-fold change in the two datasets were selected and then the intersection of the two DEG sets, which were used for the GSEA, was taken.
(3) GSEA using common DEGs: GSEA analysis was performed with the selected DEGs with the log-fold changes for SCP259. A better result may be provided when using a greater number of cells.

### 2. Experimental Animals and Tissue-Culture

Wild-type C57BL/6 mice were supplied by Samtako Bio Korea (Osan, Korea).

Primary bone marrow-derived macrophages (BMDMs) were harvested from mice and cultured in DMEM in the presence of M-CSF (R&D Systems, 416-ML) for 3 to 5 days. HEK293T cells (ATCC-11268; American Type Culture Collection) were cultured in DMEM (Gibco) containing 10% FBS (Gibco), nonessential amino acids, sodium pyruvate, streptomycin (100 μg/mL) and penicillin G (100 IU/mL).

### 3. Reagents and Antibodies

LPS (Escherichia coli O111:B4, tlrl-eblps), Adenosine 5′-triphosphate (ATP, tlrl-atpl), Nigericin and Dextran Sulfate Sodium (DSS) were purchased from Invivogen. Antibodies specific for NAMPT (ab236874) were purchased from Abcam. NAMPT (E-3), TLR4 (25), CYBB (54.1), CYBA (E-11), Actin (I-19), ASC (N-15-R), IL-18 (H-173-Y), caspase-1 p10 (M-20), HA (12CA5), Flag (D-8), GST(B-14), Myc (9E10), His (H-3) and V5 (C-9) were purchased from Santa Cruz Biotechnology. Specific Abs against IkBα (L35A5) and Phospho-IkBα (14D4) were supplied by Cell Signaling Technology (Danvers, MA, USA). IL-1β (AF-401-NA) and NLRP3 (AG-20B-0014) were used with products from R&D system and Adipogen, respectively.

### 4. Plasmid Construction

GST-NAMPT, Myc-NAMPT and Flag-TLR4 plasmids were purchased from Addgene.

The full-length (FL) CYBB and mutant plasmids were constructed as follows. Plasmids encoding different regions of NAMPT (1-491, 10-116, 188-455, 456-491) were constructed by amplifying each region from FL NAMPT cDNA by performing PCR and inserting the amplified product into a pEBG derivative including a region of encoding an N-terminal GST epitope tag between BamHI and NotI. Plasmids encoding different regions of TLR4 (1-811, 25-696, 717-811) were constructed by amplifying each region from FL TLR4 cDNA by performing PCR and inserting the amplified product into a pEF derivative including a region of encoding a C-terminal Flag tag between BamHI and NotI sites. A pEBG-GST mammalian fusion vector and a pEF-IRES-Puro expression vector were used to create all transient constructs encoded in plasmids in mammalian cells. All of the created constructions were 100% identical to the original sequence using an ABI PRISM 377 automated DNA sequencer.

### 5. Peptides

Tat-labeled NAMPT or NAMPT peptides were commercially produced and purified in an acetate salt form to circumvent undesirable responses in the cells by Peptron (Daejeon, Korea). The endotoxin level was measured by the Limulus amebocyte lysate test (Charles River Endosafe® Endochrome-K™, R1708K, Wilmington, MA, USA) and was less than 3 to 5 pg/mL at the concentrations of the peptides used in experiments.

### 6. Recombinant Protein

To obtain recombinant rCT-NAMPT proteins, sequences of NAMPT amino acids (57-65), NAMPT amino acids (52-65), and CT peptides (TWYKIAFQRNRK) were inserted to have an N-terminal 6×His tag in a pRSFDuet-1 Vector (Novagen), and the expression was induced, harvested, and purified from Escherichia coli expression in accordance with the recommended protocols by Novagen. The rVehicle, rCT, or rCT-NAMPT proteins were purified through a permeable cellulose membrane and LPS contamination was measured using a Limulus amebocyte lysate assay (Bio-Whittaker). The concentrations of the r Vehicle, rCT, or rCT-NAMPT proteins used in the experiment were adjusted to less than 20 pg/mL.

### 7. GST Pulldown, Immunoblot (IB), and Immunoprecipitation Analysis

293T and BMDM cells were subjected to GST pulldown, Western blotting, and co-immunoprecipitation analysis.

For GST pulldown, 293T cells were collected and lysed in NP-40 buffer supplemented with a protease inhibitor cocktail (Roche, Basal, CH). After centrifugation, the supernatants were pre-treated at 4° C. for 2 hours using protein A/G beads, the pre-treated lysates were combined with a 50% slurry of glutathione-conjugated Sepharose beads (Amersham Biosciences, Amersham, UK) to induce the binding reaction at 4° C. for 4 hours. The precipitates were thoroughly rinsed with a lysis buffer. Proteins conjugated to glutathione beads were eluted by heating for 5 min in a Sodium Dodecyl Sulfate (SDS) loading buffer.

Cells were collected and lysed in NP-40 buffer supplemented with a protease inhibitor cocktail (Roche, Basal, CH) for immunoprecipitation analysis. Whole-cell lysates were immunoprecipitated with the treated antibodies after pre-treated with protein A/G agarose beads for 1 hour at 4° C. In general, 1 mL of cell lysates were treated with 1 to 4 μg of antibodies for 8 to 12 hours at 4° C. After 6 hours of incubation with protein A/G agarose beads, the immunoprecipitates were thoroughly washed with the lysis buffer and eluted with an SDS loading buffer by boiling for 5 minutes.

Polypeptides were separated by SDS-polyacrylamide gel electrophoresis and then transferred to a PVDF membrane for immunoblotting (IB) (Bio-Rad, Hercules, CA, USA). For immunodetection, specific antibodies were required, and Chemiluminescence (ECL; Millipore, MA, USA) was used to visualize an antibody bound to a target. In addition, a Vilber chemiluminescence analyzer was used to detect the antibody (Fusion SL3; Vilber Lourmat, Colégien, France).

### 8. Flow Cytometry

Flow cytometry was used to quantify intracellular ROS levels. The cells were cultured in a serum-free medium and stained with 2 μM dihydroethidium (DHE for O²⁻; Calbiochem) or 1 μM 2′,7′-dichlorodihydrofluorescein diacetate (H2DCFDA for H₂O₂; Calbiochem) as a redox-sensitive dye. mAbs were incubated at 4° C. for 20 to 30 minutes to determine cell surface protein expression, and cells were fixed using a Cytofix/Cytoperm Solution (BD Biosciences), and in a specific case, mAb incubation was performed to detect intracellular proteins. The used mAb clones were the following: ITGA6 (GoH₃, BD Pharmingen™) and ITGB1 (HMβ1-1, BD Pharmingen™). The cells were washed completely and quickly with pulse spinning before being analyzed in a FACS Calibur (BD Biosciences, San Jose, CA, USA). CellQuest software (BD Biosciences) was used to visualize the data, and FlowJo software was used to analyze the data (Tree Star, Ash-land, OR, USA).

### 9. Enzyme-Linked Immunosorbent Assay

TNF-α, IL-6, IL-1β and IL-18 levels were measured in cell culture supernatants and mouse serum using the BD OptEIA ELISA system (BD Pharmingen). All experiments were performed according to the manufacturer's protocol.

10. Lentiviral shRNA Production

For silencing murine ITGA6 and ITGB1 in primary cells, pLKO.1-based lentiviral CaMKKb shRNA constructs (sc-38952-SH) and LKB1 shRNA constructs (sc-35817-SH) were obtained from Santa Cruz Biotechnology. GIPZ Lentiviral Mouse Itga6 shRNA constructs (RMM4431-200328849, RMM4431-200410349 and RMM4431-200411464) and GIPZ Lentiviral Mouse Itgb1 shRNA constructs (RMM4431-200340920, RMM4431-200350229, RMM344-2003804804806) were purchased from Open Biosystems. Lentiviruses were generated using packaging plasmids (pMDLg/pRRE, pRSV-Rev, and pMD2VSV-G) via Lipofectamine 2000-mediated transient transfection into HEK293T cells. After 72 hours of transfection, the virus-containing medium was concentrated by a ultracentrifuge. As described above, lentiviral vector titration was calculated using 293T cells, and the titration lentivirus was transduced into BMDMs.

### 11. In Vivo Lentivirus Transduction

Lentiviral particles concentrated in Method 10 above were frozen at 4° C. and diluted in PBS and polybrene (8 g/mL final concentration; Sigma) to give a dose of 1×10 pfu in a 100 L injection volume. Mice were intravenously injected with a lentivirus expressing nonspecific shRNA (shNS) or shRNA specific for ITGA6 (sh ITGA6) or ITGB1 (shITGB1) 2 times and then orally administered with DSS (Acute or chronic Colitis) and rCT-NAMPT, and then experiments were performed.

### 12. Mouse Model of Colitis

Dextran sodium sulfate (DSS)-induced acute or chronic colitis mouse models were constructed using 6-week-old C57BL/6 female mice (Samtako, Osan, Korea). To evaluate the trigger cause of acute colitis, mice were supplied with drinking water containing 3% (w/v) dextran sodium sulfate (molecular weight: 36,000 to 50,000 kDa, MP Biomedicals, Santa Ana, CA, USA). An acute colitis model was transduced with Lenti-shNS or Lenti-shITG virus (1×10¹¹ pfu/kg) on days 7 and 14 via i.v. before DSS treatment. While the mice were treated with 3% DSS for 6 days, r Vehicle, rCT or rCT-NAMPT (50 μg/kg) was i.p. injected 8 times. The survival of mice was tracked for 12 days, and mortality was measured for n=15 mice per group. The survival of the mice model transduced with Lenti-shNS had similar effects to a WT control. A chronic colitis model was treated with 3% DSS for 7 days at 3 cycles and supplied with water for 14 days in the interval of the DSS cycle. The rVehicle, rCT or rCT-NAMPT (50 μg/kg) was i.p. injected at 2 cycles together with DSS treatment. The survival of mice was tracked for 9 weeks, and mortality was measured for n=15 mice per group. To calculate causality, account for perturbations, and reduce bias, a randomization method was used to randomly assign mice to either a treatment group or a control group (or multiple intervention groups). The DSS solutions were freshly prepared every two days. The non-DSS-fed mice in the control group were supplied with sterile distilled water. The humane endpoint for body weight loss (euthanasia required) was 20% (as compared to the original body weight of an animal). Without an approved exception request, body weight loss could not exceed 20%.

### 13. Clinical Score and Histology

Body weight, degree of bloody stool, and stool concentration were measured every day during the colitis induction to obtain the clinical score. The clinical score was determined by two trained investigators who were not provided with information about the experiment. Mouse distal colon tissues were fixed in 10% formalin and embedded in paraffin for immunohistochemistry. 4 mm paraffin slices were cut and stained with hematoxylin and eosin (H&E). As mentioned above, a board-certified pathologist (Dr. Min-Kyung Kim, Seoul, Korea) independently scored each organ segment without prior knowledge of the therapy groups.

### 14. Statistical Analysis

All data are reported as mean±SD and were analyzed using the Student's t-test with a Bonferroni adjustment or ANOVA for multiple comparisons. The statistical software program SPSS (Version 12.0) was used to conduct the analysis (SPSS, Chicago, IL, USA). At p 0.05, differences were judged to be significant. Data for survival were graphed and analyzed using the Kaplan-Meier product limit method, with a log-rank (Man-tele-Cox) test for comparison in GraphPad Prism (version 5.0, La Jolla, CA, USA).

### Experiment Results

1. Confirmation of Possibility of Overexpressed eNAMPT and CYBB to Induce Chronic Inflammation in Inflammatory Bowel Disease (IBD)

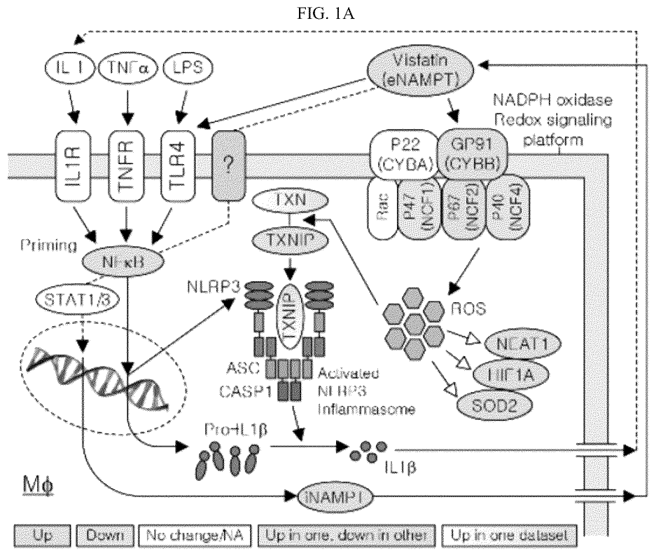

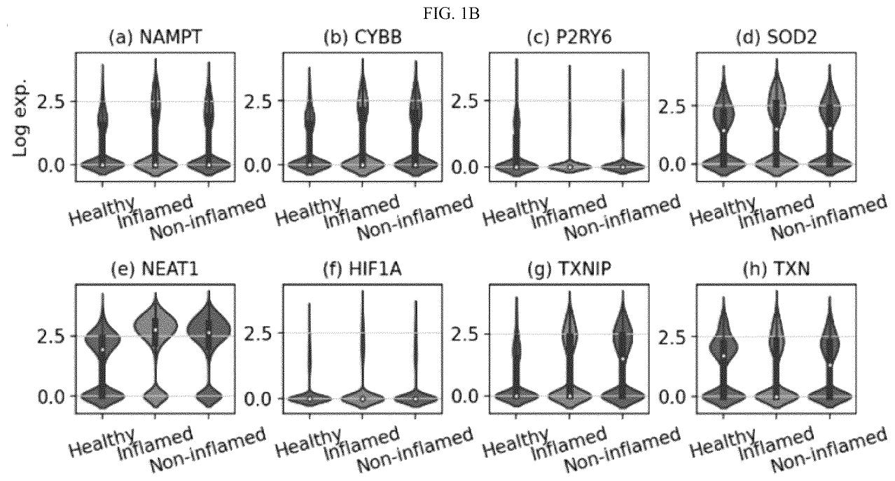

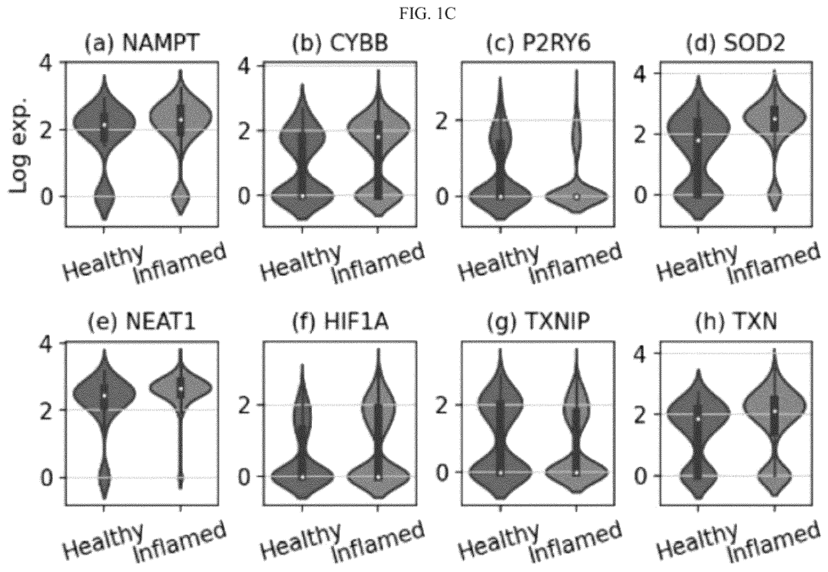

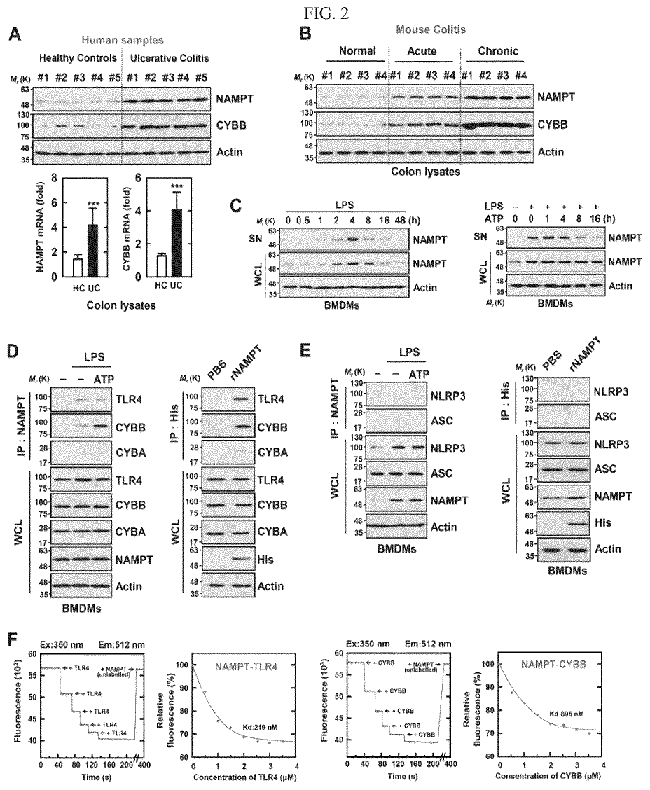

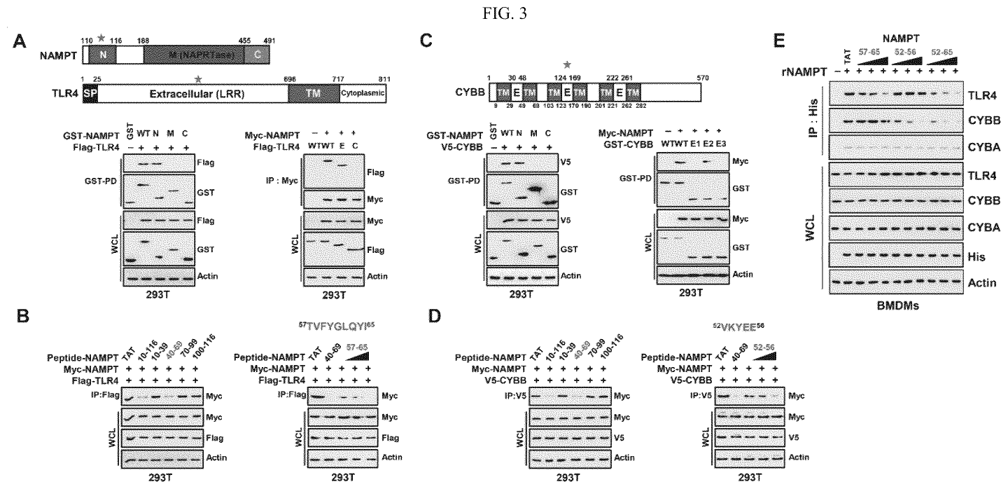

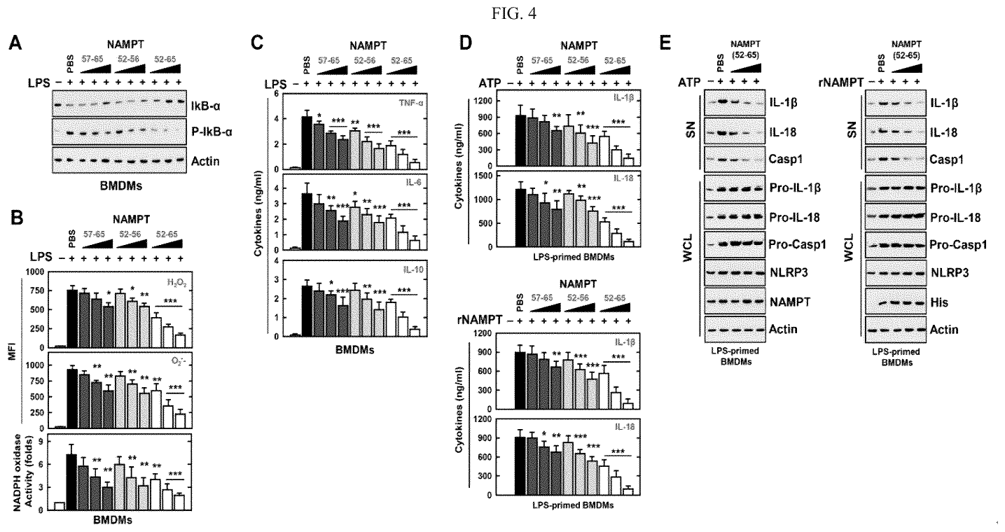

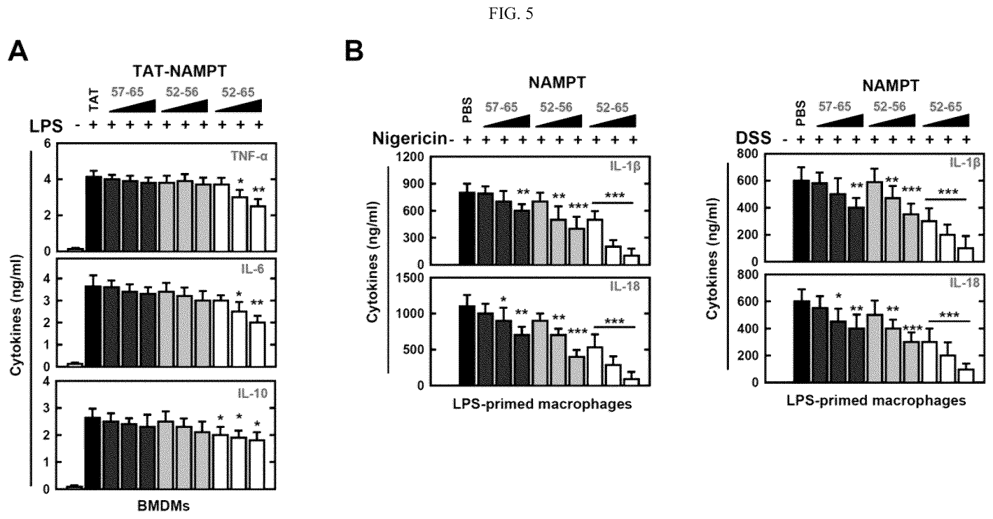

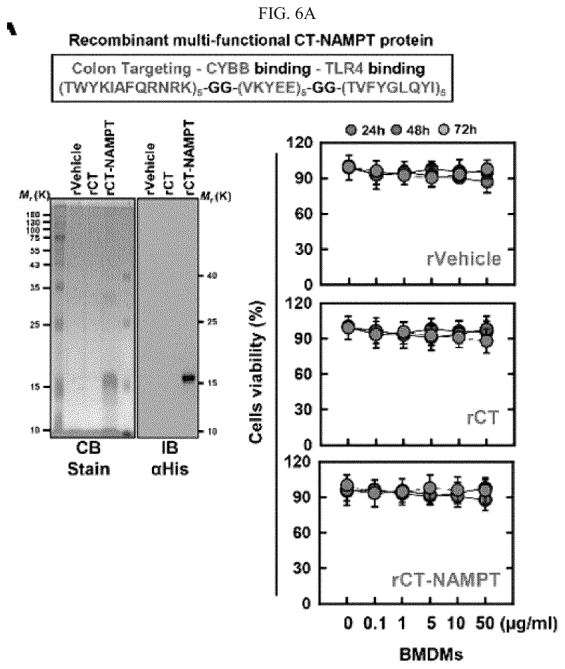

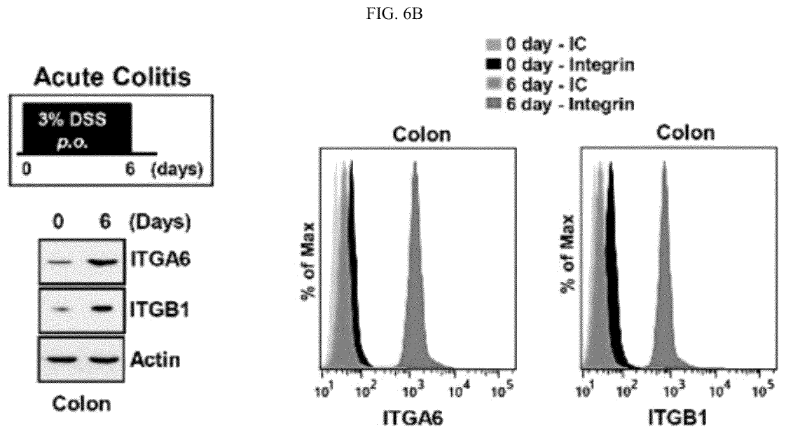

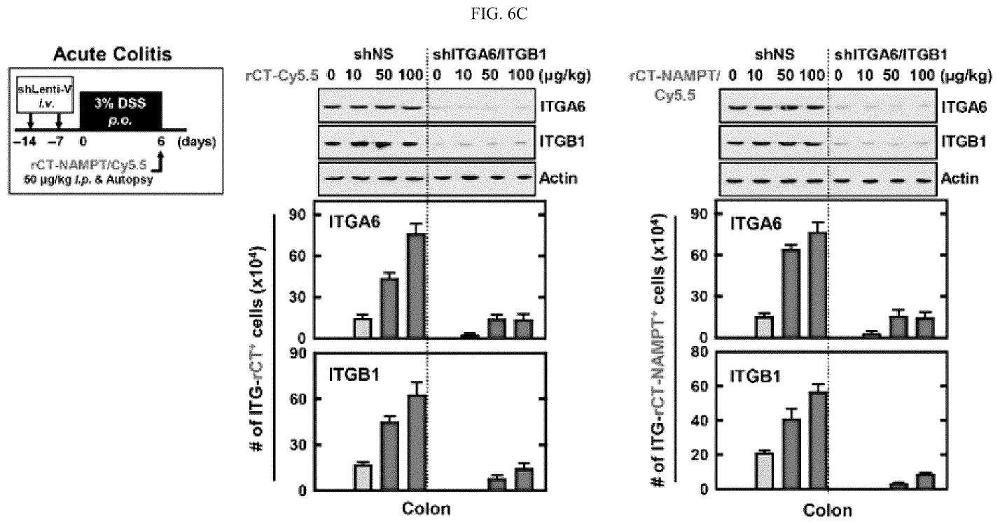

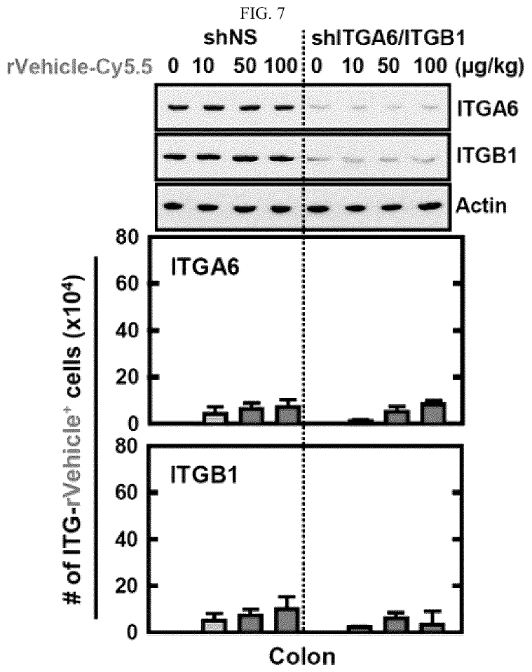

As a result of analyzing the datasets SCP259 and GSE182270, it was found that overexpressed NAMPT and CYBB (the latter also known as NOX2 and gp91Phox) may cause chronic activation of NLRP3 inflammasome in ulcerative colitis (UC) (FIG. 1A). The gastrointestinal macrophage participated in chronic inflammation in IBD, and thus, common macrophage specific DEGs were used to check the pathways using a pathway view tool. The NAMPT-CYBB interaction was associated with downstream inflammatory signaling. eNAMPT stimulated a NOX-mediated redox regulatory pathway, and NAMPT was associated with TLR4 to lead to upregulation of NLRP3 and pro-IL1β expression via an NF-κB signaling pathway (FIG. 1A). Overexpression of eNAMPT triggered an M1-skewed transcriptional program in macrophages. These study results on the overexpression of NAMPT and CYBB in inflamed tissues of patients with UC were led to analyze single-cell RNA-seq data in more detail by focusing on pathways associated with activation of the NLRP3 inflammasome through the interaction of eNAMPT with NOXs.

Table 2 shows the DEGs associated with NLRP3 inflammasome activation via the redox signaling pathway (activation), with focus on the NAMPT-NOX association and NF-κB signaling (priming). Violin plots (FIGS. 1B and 1C) showed the expression of these genes in macrophages (FIG. 1A). It may be seen that an increase in ROS via the NAMPT-NOX interaction is evident, because ROS-related genes such as SOD2, NEAT1, and HIF1A are overexpressed in inflamed UC samples (p<0.05). The percentages of macrophages expressing NAMPT were similar in inflamed and control tissues, and the expression levels were higher in inflamed samples than control samples. Although the corrected p-value for the fold-change in NAMPT expression in GSE182270 was not statistically significant, which may be explained by the relatively small number of macrophages.

TABLE 2

| | SCP259 [symbol] Macrophage [symbol] | | | | | GSE182270 [symbol] Macrophage [symbol] | | | | |
|---|---|---|---|---|---|---|---|---|---|---|
| Genes | [symbol] | [symbol] | [symbol] | [symbol] | [symbol] | [symbol] | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | [symbol] | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | [symbol] | [symbol] | [symbol] | [symbol] | [symbol] |
| [symbol] | [symbol] × 10 [symbol] | [symbol] × 10 [symbol] | [symbol] | [symbol] | [symbol] | [symbol] × 10 [symbol] | [symbol] | [symbol] | [symbol] | [symbol] |
| [symbol] | [symbol] | [symbol] | [symbol] | [symbol] | [symbol] | [symbol] × 10 [symbol] | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] | | | | | 1 | [symbol] | [symbol] | [symbol] | [symbol] |
| [symbol] | [symbol] × 10 [symbol] | [symbol] | [symbol] | [symbol] | [symbol] | [symbol] | | | | |
| [symbol] | [symbol] | 1 | [symbol] | [symbol] | [symbol] | [symbol] | | | | |
| [symbol] | [symbol] | | | | | 1 | [symbol] | [symbol] | [symbol] | [symbol] |
| [symbol] | [symbol] | 1 | [symbol] | [symbol] | [symbol] | 1 | [symbol] | [symbol] | [symbol] | [symbol] |
| [symbol] | [symbol] | 1 | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |
| [symbol] | [symbol] × 10 [symbol] | [symbol] × 10 [symbol] | [symbol] | [symbol] | [symbol] | 1 | [symbol] | [symbol] | [symbol] | [symbol] |

[symbol]

indicates data missing or illegible when filed

The gene set enrichment results (Table 3) show that in macrophages, many of the immune signals are upregulated in UC compared with those in the healthy colon. These signals include interleukins (ILs), the innate immune system, Toll-like receptor cascades, TNFα signaling via NF-κB, inflammatory responses, cytokine signaling in the immune system, and IL-4 and IL-13 signaling (FIG. 1A). These signaling events are involved in NLRP3 activation and in priming signaling pathways (FIG. 1A). Further, genes associated with IFNγ signaling, antigen processing and presentation, as well as MHC Class II antigen presentation, were downregulated. Taken together, these data indicate that eNAMPT contributes to UC and that macrophages in UC transmit higher levels of innate immune signals associated with NAMPT-CYBB-driven signaling pathways.

TABLE 3

| Cells | pathway | p.val | p.val.adj | NES |
|---|---|---|---|---|
| Macrophage | REACTOME Neutrophil Degranulation | 5.54E−06 | 0.000 | 2.347 |
| | REACTOME Signaling by Interleukins | 4.45E−05 | 0.001 | 2.270 |
| | REACTOME Innate Immune System | 2.57E−05 | 0.001 | 2.107 |
| | REACTOME Toll Like Receptor Cascades | 0.003346 | 0.012 | 1.955 |
| | HALLMARK Tnfa Signaling via Nfkb | 0.002235 | 0.009 | 1.929 |
| | HALLMARK Inflammatory Response | 0.021467 | 0.062 | 1.756 |
| | REACTOME Cytokine Signaling in Immune System | 0.006648 | 0.022 | 1.728 |
| | REACTOME Interleukin 4 and Interleukin 13 Signaling | 0.016487 | 0.052 | 1.725 |
| | WP Vitamin D Receptor Pathway | 0.021583 | 0.062 | 1.607 |
| | HALLMARK Complement | 0.024264 | 0.066 | 1.583 |
| | Class I MHC Mediated Antigen Processing Presentation | 0.04918 | 0.113 | 1.545 |
| | KEGG Leishmania Infection | 0.030507 | 0.076 | −1.630 |
| | REACTOME Interferon Gamma Signaling | 0.028175 | 0.073 | −1.698 |
| | WP Ebola Virus Pathway on Host | 0.003064 | 0.011 | −2.020 |
| | KEGG Viral Myocarditis | 0.001222 | 0.006 | −2.038 |
| | KEGG Intestinal Immune Network for Iga Production | 0.001302 | 0.006 | −2.076 |
| | KEGG Asthma | 0.000962 | 0.005 | −2.125 |
| | KEGG Allograft Rejection | 0.000921 | 0.005 | −2.127 |
| | KEGG Autoimmune Thyroid Disease | 0.000921 | 0.005 | −2.127 |
| | KEGG Graft Versus Host Disease | 0.000921 | 0.005 | −2.127 |
| | KEGG Type I Diabetes Mellitus | 0.000921 | 0.005 | −2.127 |
| | KEGG Antigen Processing and Presentation | 0.000349 | 0.003 | −2.187 |
| | REACTOME Mhc Class II Antigen Presentation | 0.000368 | 0.003 | −2.188 |
| | KEGG Systemic Lupus Erythematosus | 5.97E−05 | 0.001 | −2.313 |
| | WP Allograft Rejection | 4.28E−05 | 0.001 | −2.333 |
| Cytotoxic T cell | HALLMARK Interferon Gamma Response | 0.022948 | 0.172 | −1.751 |
| | REACTOME Signaling by Gpcr | 0.002779 | 0.041 | −1.896 |
| | HALLMARK Il2 Stat5 Signaling | 0.002974 | 0.041 | −1.963 |
| | HALLMARK Tnfa Signaling via Nfkb | 0.004098 | 0.041 | −1.969 |
| CD4 T cells | HALLMARK Allograft Rejection | 0.037225 | 0.181 | −1.583 |
| | HALLMARK Apoptosis | 0.012435 | 0.069 | −1.856 |
| | WP Il18 Signaling Pathway | 0.011749 | 0.069 | −1.868 |
| | REACTOME Neutrophil Degranulation | 0.005372 | 0.066 | −1.874 |
| | HALLMARK Interferon Gamma Response | 0.008441 | 0.066 | −1.895 |
| | REACTOME Signaling by Gpcr | 0.007646 | 0.066 | −1.910 |
| | HALLMARK Inflammatory Response | 0.000406 | 0.008 | −2.188 |
| | HALLMARK Tnfa Signaling via Nfkb | 1.28E−06 | 0.000 | −2.707 |
| Regulatory T cell | REACTOME Signaling by Gpcr | 0.009243 | 0.025 | −1.858 |
| | HALLMARK Hypoxia | 0.00407 | 0.016 | −1.958 |
| | HALLMARK Tnfa Signaling via Nfkb | 0.000606 | 0.005 | −2.180 |

* p.val: p-value, p.val.adj: adjusted p-value

2. Confirmation of Interaction Between CYBB and TLR4 of eNAMPT

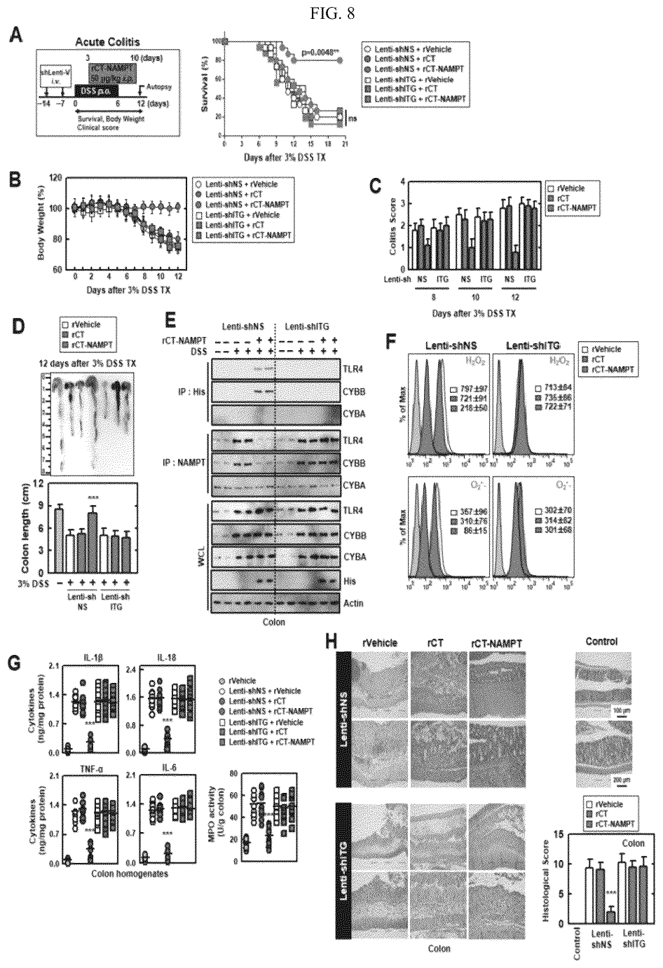

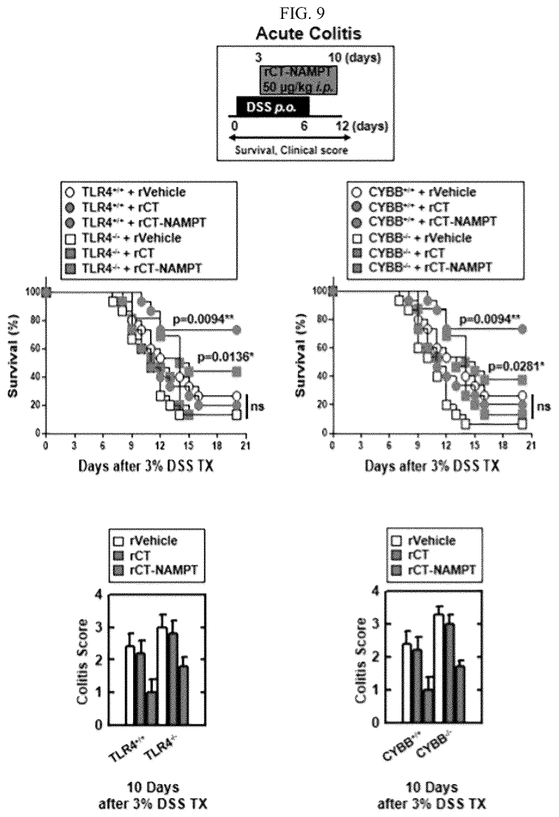

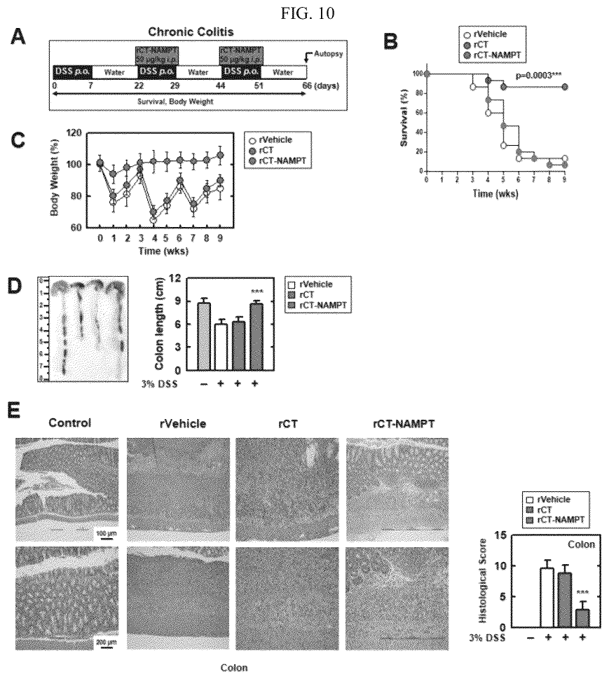

Next, NAMPT and CYBB expression was further analyzed in colon tissue sections from normal subjects and patients with UC. The mRNA and protein levels of NAMPT and CYBB were approximately 3 to 4 times higher in the colon tissues of patients with UC than those of a control group (FIG. 2A). Further, the expression of NAMPT and CYBB remarkably increased in association with increased disease severity in normal mice or mice with acute or chronic colitis (FIG. 2B).

To confirm a role for eNAMPT in activating the NLRP3 inflammasome in macrophages, it was examined whether the interaction of eNAMPT with TLR4 or CYBB was involved. A two-signal model was proposed to explain activation of the inflammasome by NLRP3. Signal 1 is a priming signal created by microbial components or endogenous cytokines that mediate NF-κB activation and a subsequent increase of NLRP3 and prointerleukin-1β levels. Various molecules, including extracellular ATP and pore-forming toxins, transmit inflammatory activation signals (signal 2). Several molecular or cellular events activate the NLRP3 inflammasome, such as ion flux, mitochondrial dysfunction, ROS generation, and lysosomal damage.

First, it was found that NAMPT expressed by TLR4 (LPS) or an NLRP3 inflammasome inducer (LPS/ATP) increased intracellular (i) NAMPT expression and markedly increased extracellular (e) NAMPT expression in macrophages (FIG. 2C). Further, eNAMPT interacted with TLR4 or CYBB in the presence of LPS; and eNAMPT (rNAMPT) treated with recombinant NAMPT protein interacted with TLR4 or CYBB in macrophages. In contrast, the NAMPT interaction with CYBA was negligible in macrophages (FIG. 2D). Further, an interaction between NAMPT and NLRP3 or ASC was undetectable (FIG. 2E). Furthermore, as a result evaluated by a fluorescence binding experiment with recombinant proteins (rNAMPT) and a fluorescence labeling experiment using TLR4 or CYBB with NAMPT, the in vitro interaction between NAMPT and TLR4 or CYBB exhibited a sufficiently high affinity (TLR4, 219 nM; CYBB, 896 nM) (FIG. 1). Together, these results suggest that the interaction of eNAMPT with TLR4 or CYBB mediates the activation of the NLRP3 inflammasome in inflammatory colitis, which indicates the potential clinical importance of these events.

### 3. NAMPT Amino Acid Sequence Required for Binding of TLR4 and CYBB

To identify the amino acid (aa) residues in NAMPT that interact with TLR4 and CYBB, vectors were constructed to express full-length and mutant NAMPT, TLR4, and CYBB. NAMPT consists of N-terminal, middle (NAPRTase), and C-terminal domains (FIG. 3A). To identify the domain required for the interaction between NAMPT and TLR4, a construct tagged with GST or Myc-NAMPT and Flag-TLR4 was used. In 293T cells, the N-terminus of NAMPT bound TLR4 and the extracellular leucine-rich region (LRR domain) of TLR4 were essential for the interaction with NAMPT (FIG. 3A).

The cell-penetrating TAT peptide (GRKKRRQRRRPQ) overcomes the lipophilic barrier of cellular membranes and thus delivers large molecules as well as small particles into the cell to exert their activities. To investigate in detail the sequence of the N-terminus of NAMPT, TAT-NAMPT peptides (separated by 20 aa) included in aa 10-116 of NAMPT were constructed. The TAT-NAMPT peptides were incubated with Myc-NAMPT and Flag-TLR4 expressed in 293T cells, and the complexes were immunoprecipitated (IP) with a Flag antibody. Treatment with TAT-NAMexemPT (aa 40-69) reduced the binding of NAMPT to TLR4, which indicates that this region binds to TLR4 (left of FIG. 3B). To determine the minimum aa sequence necessary for NAMPT-TLR4 binding, the PredictProtein software (https://predictprotein.org; accessed on Jul. 9, 2022) was used to predict elements of protein function and structure using database searches, homology_based inference, machine learning, and artificial intelligence. PredictProtein predicted that aa 57-65 of NAMPT were required for binding TLR4, consistent with results that NAMPT-TLR4 binding in 293T cells was reduced by TAT-NAMPT (aa 57-65) in a concentration-dependent manner (right of FIG. 3B).

Next, we investigated the region of NAMPT that bound to CYBB in 293T cells. The N-terminus of NAMPT bound to CYBB, and extracellular domain 2 of CYBB were required for interaction with NAMPT (FIG. 3C). Further, aa 40-69 of NAMPT, the TLR4 binding residues, bound to CYBB (left of FIG. 3D). PredictProtein predicted that NAMPT aa 52-56, as aa binding to CYBB, consistent with the decrease in NAMPT-CYBB binding in 293T cells co-expressing TAT-NAMPT (aa 52-56) in a concentration-dependent manner (right of FIG. 3D).

Next, it was tested whether TAT-NAMPT aa 57-65 or aa 52-56 had inhibited binding of eNAMPT to TLR4 or CYBB in macrophages. Consistent with the iNAMPT-TLR4 blockade by TAT-NAMPT (aa 57-65) and the iNAMPT-CYBB blockade by TAT-NAMPT (aa 52-56), TAT-NAMPT (aa 57-65) specifically inhibited eNAMPT-TLR4 binding, and TAT-NAMPT (aa 52-56) specifically inhibited eNAMPT-CYBB binding (FIG. 3E). Moreover, TAT-NAMPT (aa 52-65) inhibited the eNAMPT-TLR4 and eNAMPT-CYBB binding interactions. These results show that NAMPT aa 57-65 and aa 52-56 are necessary for interacting with TLR4 or CYBB, respectively, and show that NAMPT interactions are genetically separated.

### 4. Confirmation of Inhibition of NLRP3 Inflammasome Activation by NAMPT Peptide

To determine the effect of NAMPT peptides related to signal 1 for the NLRP3 inflammasome in macrophages, LPS-treated BMDMs were exposed to different concentrations of NAMPT peptides (aa 57-65, aa 52-56, or aa 52-65). NAMPT peptides aa 57-65 and aa 52-56 partially inhibited the activation of NF-κB, generation of ROS, and production of cytokines. However, NAMPT peptides aa 52-65 markedly inhibited the production of ROS and NF-κB-induced cytokine (FIGS. 4A to 4C). Further, TAT-NAMPT peptides aa 52-65 other than TAT-NAMPT peptides aa 57-65 and aa 52-56 partially inhibited NLRP3 inflammasome activation signal 1 (FIG. 5A). Thus, the TAT-NAMPT peptides were less likely to inhibit the interaction of eNAMPT with TLR4 or CYBB.

Accordingly, it was examined whether NAMPT peptides had a special role in the modulation of signal-2 activation of the NLRP3 inflammasome. It was found that NAMPT peptide aa 52-65 efficiently inhibited the maturation of IL-1β and IL-18, as well as ATP-induced caspase-1 cleavage and nigericin or DSS stimulation (FIG. 4D upper, FIG. 4E left and FIG. 5B). It was found that the NAMPT peptide aa 52-65 specifically inhibited the action of eNAMPT (FIG. 4D lower, FIG. 4E right). These study results indicate that actions of NAMPT in the TLR4-mediated signaling pathway and in CYBB-containing NOXs are functionally and genetically separable. Together, these data provide evidence that the NAMPT peptide aa 52-65 is an essential negative regulator of signal 1 and signal 2 in response to NLRP3 inflammasome activation.

### 5. Confirmation of Colon Targeting of Recombinant Multifunctional CT-NAMPT

The experiments presented above indicate that NAMPT peptides (aa 57-65 and aa 52-56) directly blocked the binding of eNAMPT to the extracellular domains of CYBB and TLR4 and subsequently attenuated the activation of the NLRP3 inflammasome in macrophages.

A peptide TWYKIAFQRNRK (abbreviated TK), derived from a COOH-terminal globular domain of laminin-1 al chain, was used as a vehicle for targeted drug delivery to the colon. Thus, TK interacts with integrin α6β1, with high affinity for colonic tissue. Accordingly, it was developed a recombinant 12-residue TK peptide (CT) conjugated to a multifunctional NAMPT (rCT-NAMPT) in which CT targets the colon and has the essential and minimal aa residues required for CYBB/TLR4 binding. The authenticity of the predicted product was confirmed using SDS-polyacrylamide gel electrophoresis and immunoblotting (left of FIG. 6A). There were no significant differences compared with the vehicle control associated with rCT-NAMPT-induced cytotoxicity in BMDMs (right of FIG. 6A).

A mouse model of DSS-induced colitis was used to further investigate the physiological significance of rCT-NAMPT in inflammatory colitis (FIGS. 6B and 6C). It was found that the expression of ITGA6 (integrin α6) and ITGB1 (integrin β1), which bound to TK peptides (CT), was significantly increased in the colon of mice with acute colitis (FIG. 6B). Next, to evaluate the specificity of rCT-NAMPT, ITGA6- or ITGB1-knockdown mice were generated through sh-Lentiviral transduction. The mice were treated with DSS and administered rCT-NAMPT/Cy5.5 via intraperitoneal injection on day 6. The rCT or rCT-NAMPT specifically targeted colonic tissues in mice with acute colitis, but not in other organs (FIGS. 6C and 7, and data not shown). These results show that rCT binds to colonic tissues, which raises the possibility of designing colon-targeted drug delivery systems for use as pharmaceutical applications for treating DSS-induced acute colitis.

6. Confirmation of rCT-NAMPT Activity of Acute and Chronic DSS-Induced Colitis Animal Model

Next, the medicinal effects of rCT-NAMPT on mouse models were evaluated in DSS induced acute and chronic colitis. For this purpose, ITG (ITGA6 and ITGB1)-knockdown mice were generated through sh-Lentiviral transduction on days 7 and 14 via i.v., before DSS treatment. While the mice were treated with 3% DSS for 6 days, rVehicle, rCT or rCT-NAMPT (50 μg/kg) was i.p. injected 8 times. rCT-NAMPT significantly increased the survival rates of Lenti-shNS-transduced mice with DSS-induced colitis, but not those of Lenti-shITG-transduced mice. Neither rVehicle or rCT significantly affected mortality, which suggests that NAMPT peptides contribute to the regulation of the inflammatory response (FIG. 8A). Furthermore, body-weight loss in Lenti-shNS-transduced mice treated with rCT-NAMPT was reduced by about 20% compared to rVehicle- or rCT-treated mice. Body weights of Lenti-shITG-transduced mice were higher than in Lenti-shNS-transduced mice, but there was no significant difference (FIG. 8B).

The colitis scores of mice were markedly decreased in Lenti-shNS-transduced mice treated with rCT-NAMPT (FIG. 8C).

After 12 days, the length of the colon, which was an indication of colitis, was measured. The colon length was recovered in rCT-NAMPT-treated Lenti-shNS-transduced mice, but remained unchanged in Lenti-shITG-transduced mice (FIG. 8D).

Further, it was tested whether rCT-NAMPT exerted pharmacological activity in vivo. For example, the in vivo detection of interactions between the NAMPT and TLR4-mediated signaling pathways and CYBB-containing NOXs may prove to be important for the evaluation of rCT-NAMPT to identify drugs that treat lethal inflammatory disease. For this purpose, the binding of NAMPT, ROS levels, and cytokine production was analyzed in the colon. The interaction of NAMPT with TLR4 or CYBB was detected only in the colon of DSS-treated Lenti-shNS-transduced mice, but not detected in Lenti-shITG-transduced mice (FIG. 8E). Cellular ROS levels decreased in Lenti-shNS-transduced mice treated with rCT-NAMPT, but not reduced in Lenti-shITG-transduced mice (FIG. 8F).

Moreover, the activity of myeloperoxidase involved in the production of TNF-α, IL-1β, IL-6, and IL-18 and the activity of NLRP3 inflammasome were measured (FIG. 8G). The histological scores of colitis (H&E staining) revealed that the rVehicle- and rCT-treated colon was disrupted by DSS in Lenti-shNS-transduced mice, and the addition of rCT-NAMPT restored the colonic barrier. Further, ITGA6/ITGB1-knockdown had no effect on rCT-NAMPT because of loss of colon-targeting ability (FIG. 8H). Particularly, rCT-NAMPT had a partially therapeutic effect against acute DSS-induced colitis in TLR4^{-/-} or CYBB^{-/-} mice compared with WT mice (FIG. 9).

Thereafter, the therapeutic effect of rCT-NAMPT on chronic colitis was investigated and mice were administered with DSS and rCT-NAMPT for 66 days (FIG. 10A). rCT-NAMPT, like acute colitis, increased mouse survival rates by about 90%. (FIG. 10B). Body weights fluctuated in the rVehicle- and rCT-treated mice, although rCT-NAMPT-treated mice maintained their body weights (FIG. 10C). Further, the lengths of the colons of rCT-NAMPT-treated mice were restored compared with those of DSS-treated mice, which were significantly damaged (FIG. 10D). H&E colon examination demonstrated that rCT-NAMPT dramatically improved colon vitality in DSS-treated mice (FIG. 10E). These study results suggest that rCT-NAMPT has a therapeutic effect against DSS-induced colitis by inhibiting the NAMPT-TLR4 or -CYBB interaction in vivo.

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

1. A pharmaceutical composition for preventing or treating inflammatory bowel disease comprising a nicotinamide phosphoribosyltransferase (NAMPT)-derived peptide as an active ingredient,
wherein the NAMPT consists of SEQ ID NO: 1, and
the NAMPT-derived peptide comprises at least one amino acid sequence selected from the group consisting of amino acids 57 to 65, amino acids 52 to 56, and amino acids 52 to 65 of NAMPT.

2. The pharmaceutical composition of claim 1, wherein the NAMPT-derived peptide is a laminin α-1-derived peptide bound to an N-terminus or C-terminus.

3. The pharmaceutical composition of claim 2, wherein the laminin α-1-derived peptide comprises an amino acid sequence represented by SEQ ID NO: 2.

4. The pharmaceutical composition of claim 1, wherein the NAMPT-derived peptide binds to TLR4 or CYBB.

5. The pharmaceutical composition of claim 1, wherein the NAMPT-derived peptide inhibits activation of NLRP3 inflammasome.

6. The pharmaceutical composition of claim 1, wherein the composition reduces NLRP3-mediated inflammation.

7. A health functional food composition for preventing or improving inflammatory bowel disease comprising a nicotinamide phosphoribosyltransferase (NAMPT)-derived peptide as an active ingredient,
wherein the NAMPT consists of SEQ ID NO: 1, and
the NAMPT-derived peptide comprises at least one amino acid sequence selected from the group consisting of amino acids 57 to 65, amino acids 52 to 56, and amino acids 52 to 65 of NAMPT.
